(19) United States
(12) Patent Application Publication
(10) Pub. No.: US 2022/0002654 A1
(43) Pub. Date: Jan. 6, 2022

(54) LARGE-SCALE CELL CULTURE SYSTEM

(71) Applicant: AMOGREENTECH CO., LTD., Gimpo-si, Gyeonggi-do (KR)

(72) Inventors: Seon Ho JANG, Seoul (KR); Kyung Gu HAN, Goyang-si, Gyeonggi-do (KR); In Yong SEO, Seoul (KR); Dong Sik SEO, Incheon (KR); Hee Sung PARK, Seoul (KR)

(73) Assignee: AMOGREENTECH CO., LTD., Gimpo-si, Gyeonggi-do (KR)

(21) Appl. No.: 17/293,741

(22) PCT Filed: Nov. 14, 2019
(86) PCT No.: PCT/KR2019/015499

§ 371 Date: May 13, 2021

(30) Foreign Application Priority Data
Nov. 14, 2018 (KR) 10-2018-0140008
Nov. 13, 2019 (KR) 10-2019-0145044

(51) Int. Cl. C12M 1/00; C12M 1/12; C12M 3/00; C12M 3/04; C12M 3/06

(52) U.S. Cl. CPC C12M 41/14; C12M 25/02; C12M 29/00; C12M 27/14; C12M 23/48; C12M 27/10; C12M 23/34

(57) ABSTRACT

A large-scale cell culture system is provided. The large-scale cell culture system according to one embodiment of the present invention includes: an incubator that includes an inner space to provides a culture environment in which a cell is stably cultured; a cell culture part that is disposed in the inner space and includes multiple supporters for cell culture disposed therein; a medium supply part that is disposed in the inner space and stores a predetermined amount of medium supplied to the cell culture part; and a pump that is disposed in the inner space, and is connected to the cell culture part and the medium supply part through a connection pipe, respectively, and circulates the medium so that the medium stored in the medium supply part is recovered to the medium supply part after being supplied to the cell culture part, the multiple supporters being provided in a plate shape having a predetermined area, and arranged in a state separated apart from each other at a predetermined distance along a height direction inside the cell culture part.

### TECHNICAL FIELD

This invention relates to a cell culture system, and more specifically, to a large-scale cell culture system capable of culturing a large number of cells through a single process.

### BACKGROUND ART

A cell culture method is a method culturing or proliferating cells by removing a tissue piece from an individual of a multicellular organism and supplying nutrients to the removed tissue piece in a container.

An animal cell culture technology is taking an important role in the industrialization of biopharmaceuticals in the field of biotechnology that has been rapidly developing since the 1980s. Accordingly, the importance of mass cultivation of animal cells began to emerge from the mid-1980s.

The animal cells derived from human or animal tissues may be suspended in a medium or cultured while attached to a carrier. Mainly, the cells derived from blood cells (including hematopoietic stem cells) are floating cells, and the cells derived from tissues such as skin, liver or lung, and embryonic stem cells or mesenchymal stem cells are adherent cells. The floating cells can proliferate in a state in which the cells are alone suspended in a medium, but adherent cells can proliferate only in a state attached to the surface of a supporter.

Accordingly, when the cells are scaled up, the floating cells are advantageous to maintain the highest cell density per unit volume, so the mass culture method of cells has mainly been made for the floating cells, but the development of a method or device for mass culturing for adherent cells is insufficient.

### DETAILED DESCRIPTION OF THE INVENTION

### Technical Problem

This invention was devised in consideration of the points, an object of the present invention is to provide a large-scale cell culture system capable of culturing adherent cells in large quantities through a single process.

### Technical Solution

In order to solve the problems, the present invention provides a large-scale cell culture system including: an incubator that includes an inner space to provides a culture environment in which a cell is stably cultured; a cell culture part that is disposed in the inner space and includes multiple supporters for cell culture disposed therein a medium supply part that is disposed in the inner space and stores a predetermined amount of medium supplied to the cell culture part; and a pump that is disposed in the inner space, and is connected to the cell culture part and the medium supply part through a connection pipe, respectively, and circulates the medium so that the medium stored in the medium supply part is recovered to the medium supply part after being supplied to the cell culture part, the multiple supporters being provided in a plate shape having a predetermined area, and arranged in a state separated apart from each other at a predetermined distance along a height direction inside the cell culture part.

At this time, the supporter may include a plate-shaped nanofiber membrane that is coated with a protein motif, and a support member that is attached to one surface of the nanofiber membrane through an adhesive layer to support the nanofiber membrane. As another example, the supporter may be a plasma-treated plate-shaped film member.

In addition, the inner space may be formed as one inner space in which a temperature is maintained at a constant temperature while a carbon dioxide concentration is maintained at a certain level, and the cell culture part and the medium supply part may be disposed together in the one inner space.

Alternatively, the incubator may include a constant temperature chamber that maintains a temperature of the inner space at a constant temperature, and in which the cell culture part and the pump are disposed, and a carbon dioxide supply chamber that is disposed inside the constant temperature chamber and maintains the internal carbon dioxide concentration at a certain level, and the medium supply part may be disposed inside the carbon dioxide supply chamber.

In addition, the cell culture part may include an enclosure-shaped culture housing having an accommodation space filled with the medium, the multiple supporters that are arranged in multiple stages in the accommodation space so that a cell is cultured, and a separated member that separates between two supporters facing each other so that the multiple supporters are separated apart from each other along a height direction of the culture housing.

At this time, the separated member may be configured in various ways. As one example, the separated member may be configured with multiple supporting bars and a spacer or multiple guide members on which a slot groove is formed. Besides, the separated member may be configured in a combination of the supporting bar, the spacer and the guide member.

In addition, a distributing plate may be disposed between the medium inlet and the supporter disposed in the accommodation space so that the medium introduced through the medium inlet is dispersed.

In addition, the medium supply part may include a medium housing that is in an enclosed shape with an open upper portion, and has a storage space in which a certain amount of the medium is stored, and an inlet and an outlet through which the medium flows in or out so that the medium is returned to the storage space or supplied to the cell culture part, and the inlet may be formed in the medium housing so as to be located at a position relatively higher than the outlet. In this case, the inlet and outlet may be formed on the opposite surface of the medium housing, respectively.

Alternatively, the medium housing may include at least one partition wall that protrudes from a bottom surface of the medium housing so that one end is connected to an inner surface of the medium housing and the other end is separated apart from the other inner surface facing the inner surface of the medium housing, and the storage space may be divided into a medium recovery space connected to the inlet and a medium supply space connected to the outlet through the partition wall. In this case, the inlet and the outlet may be formed on the same surface of the medium housing, respectively.

On the other hand, the medium supply part may include a filter member that covers the open upper portion of the medium housing, and the carbon dioxide may be supplied to the medium stored in the storage space after passing through the filter member.

In addition, the large-scale cell culture system may further include at least one driving part that rotates the cell culture part. In this case, the cell culture part may be installed in the inner space of the incubator to enable both a first rotation around an X-axis and a second rotation around a Z-axis through driving of the driving part.

### Advantageous Effects

According to the present invention, it is possible to improve productivity, reduce cost, and maintain uniformity of quality by cultivating a large number of cells simultaneously through a single process.

### BRIEF DESCRIPTIONS OF DRAWINGS

FIG. 1 is a schematic diagram showing a large-scale cell culture system according to an embodiment of the present invention,

FIG. 2 is a schematic diagram showing a large-scale cell culture system according to another embodiment of the present invention,

FIG. 3 is a diagram showing a cell culture part that can be applied to the large-scale cell culture system according to the present invention,

FIG. 4 is the exploded diagram of FIG. 3,

FIG. 5 is a cross-sectional diagram of FIG. 3 in a A-A direction,

FIG. 6 is a diagram showing another type of a cell culture part that can be applied to the large-scale cell culture system according to the present invention,

FIG. 7 is the exploded diagram of FIG. 6,

FIG. 8 is a cross-sectional diagram of FIG. 6 in a B-B direction,

FIG. 9 is a cross-sectional diagram of FIG. 6 in a C-C direction,

FIG. 10 is a variation example of FIG. 6,

FIG. 11 is the exploded diagram of FIG. 10,

FIG. 12 is a cross-sectional diagram of FIG. 10 in a D-D direction,

FIG. 13 is a diagram showing another type of a cell culture part that can be applied to the large-scale cell culture system according to the present invention,

FIG. 14 is the exploded diagram of FIG. 13,

FIG. 15 is the combined cross-sectional diagram of FIG. 13,

FIG. 16 is a diagram showing a supporter that can be applied to the cell culture part according to the present invention,

FIG. 17 is a view showing another type of a supporter that can be applied to the cell culture part according to the present invention,

FIG. 18 is a diagram showing a distributing stand that can be applied to the cell culture part according to the present invention,

FIG. 19 is a diagram showing a medium supply part that can be applied to the large-scale cell culture system according to the present invention,

FIG. 20 is the combined cross-sectional diagram of FIG. 19 in E-E direction,

FIG. 21 is a diagram showing another type of a medium supply part that can be applied to the large-scale cell culture system according to the present invention,

FIG. 22 is a plane diagram of the medium housing in FIG. 21,

FIG. 23 is a schematic diagram showing the configuration of a driving part that can be applied to the large-scale cell culture system according to the present invention,

FIG. 24 is a schematic diagram showing the method in which the cell culture part rotates around the X-axis in FIG. 23,

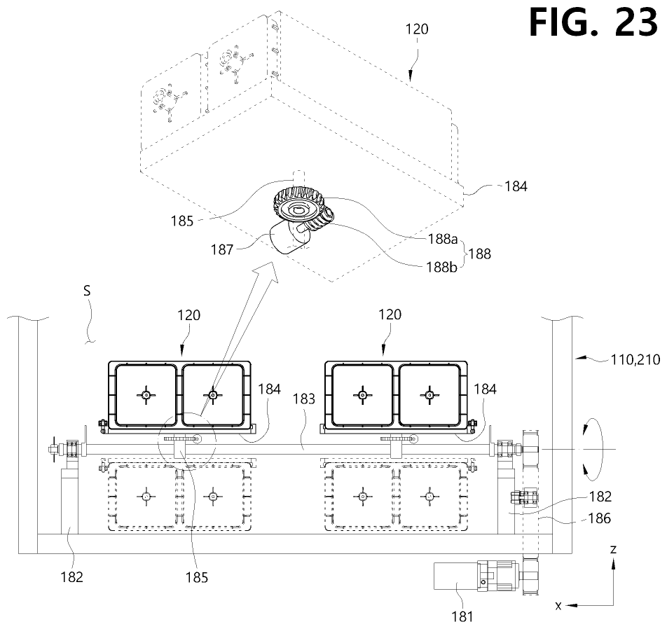

and FIG. 25 is a schematic diagram showing the method in which the cell culture part rotates around the Z-axis in FIG. 23.

### THE MODE FOR THE IMPLEMENTATION OF THE INVENTION

Hereinafter, referring to the attached drawing, embodiments of the present invention will be described in detail so that those of ordinary skills in the technical field to which the present invention belongs can easily implement. The present invention may be implemented in various different forms, and is not limited to the embodiments described herein. In the drawings, parts irrelevant to the description are omitted in order to clearly describe the present invention, and the same reference marks are added to the same or similar elements throughout the specification.

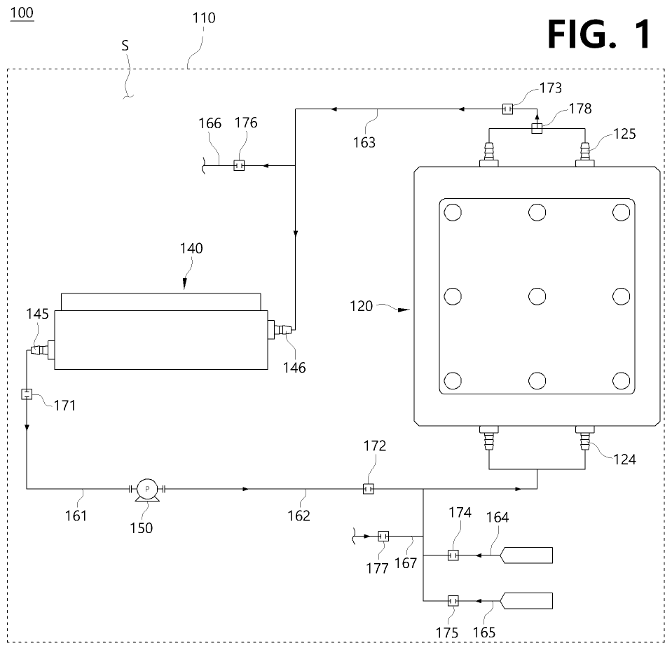

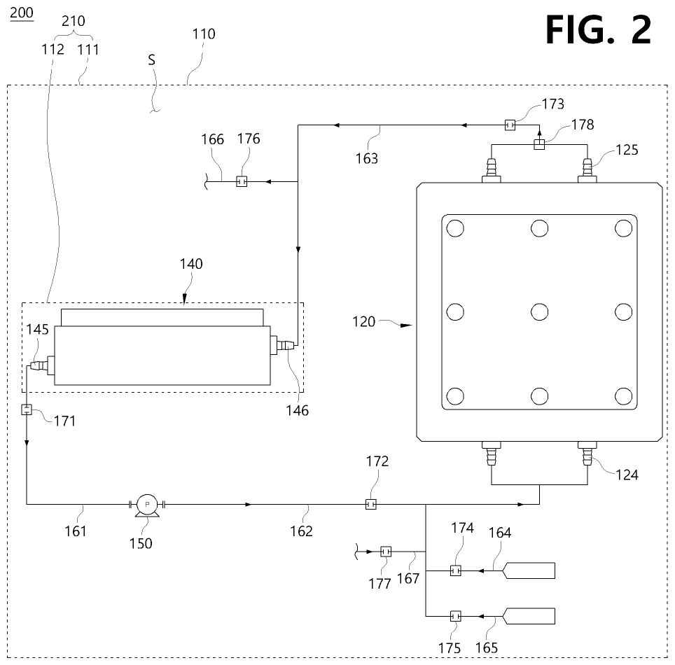

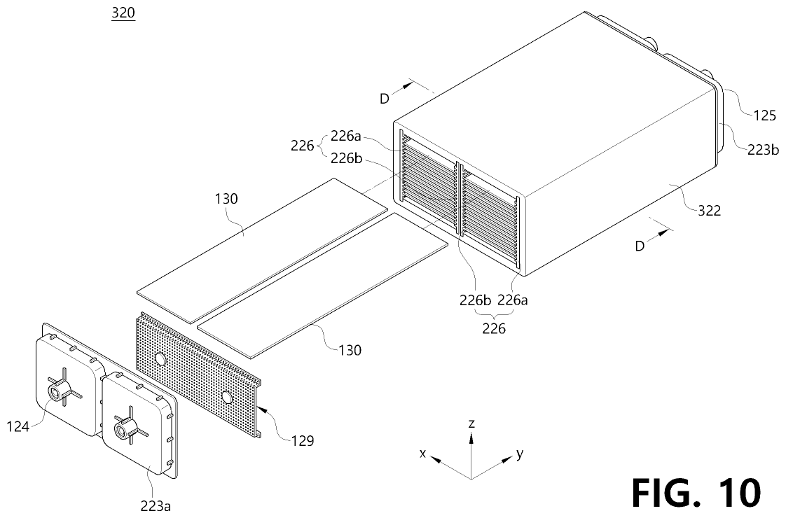

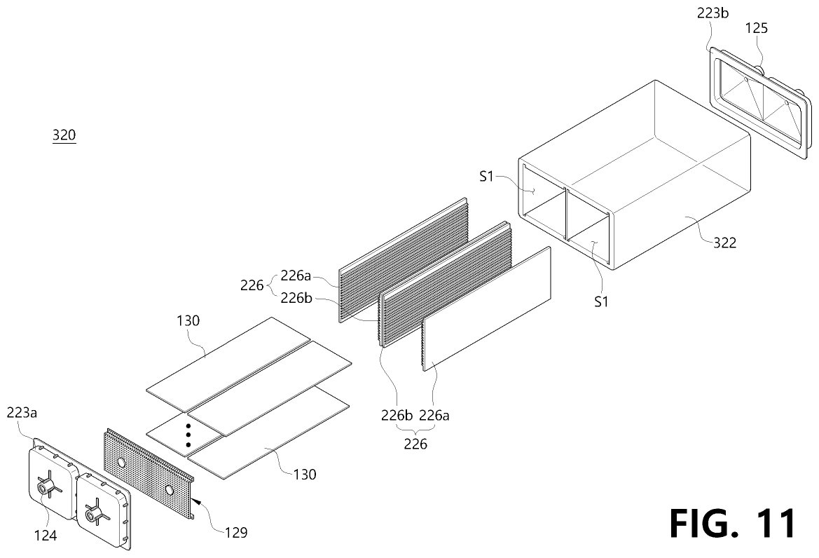

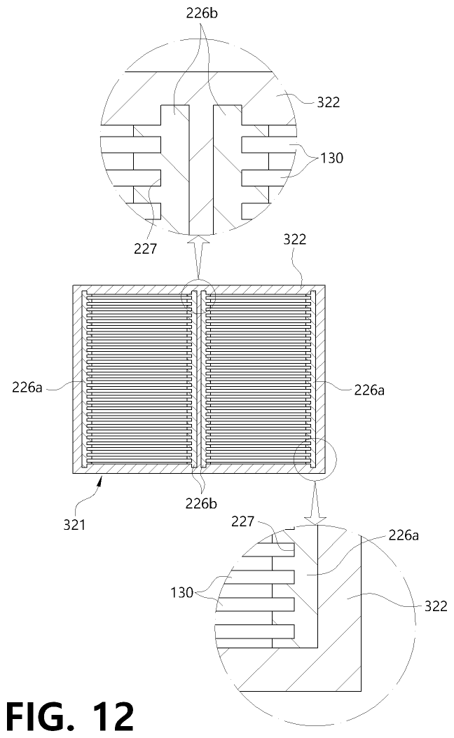

As shown in FIG. 1, FIG. 2, FIG. 10 to FIG. 15, the large-scale cell culture system (100,200) according to embodiments of the present invention includes an incubator (110,210), a cell culture part (120,220,320,420), a medium supply part (140,240) and a pump (150).

The incubator (110,210) may provide a culture environment in which the cells attached to the supporters (130,230) can be stably cultured.

As an example, the incubator (110,210) may be a chamber having an inner space (S), and the inner space (S) may be a space in which a temperature is maintained constant and a concentration of carbon dioxide is also maintained at constant concentrations.

Here, the incubator (110,210) may include an air conditioning system that maintains the temperature of the inner space (S) at a constant temperature, and may further include a carbon dioxide supply means (not shown) that supplies the carbon dioxide to the inner space (S).

Accordingly, carbon dioxide may be flowed from the inner space (S) to the medium supply part (140, 240) disposed in the inner space (S), and the carbon dioxide flowed from the inner space (S) may be dissolved in the medium stored in the medium supply part (140, 240).

Accordingly, the medium supply part (140,240) can stably supply a medium having a constant PH to the cell culture part (120,220,320,420), and the cells included in the cell culture part (120,220,320,420) may be cultured smoothly.

At this time, the incubator (110) may be a chamber having one inner space (S) as shown in FIG. 1. In this case, the inner space (S) can be maintained at a constant temperature through the above-mentioned air conditioning system, and the concentration of carbon dioxide may be maintained at a certain level through the carbon dioxide supply means. In addition, the cell culture part (120,220,320,420), the medium supply part (140, 240) and the pump (150) may be appropriately disposed in the inner space (S).

Alternatively, the incubator (210) may be composed of two chambers as shown in FIG. 2. That is, the incubator (210) may be composed of a constant temperature chamber (111) that maintains the temperature of the inner space (S) at constant temperature and a carbon dioxide supply chamber (112) that is disposed inside the constant temperature chamber (111) and maintains the internal carbon dioxide concentration at a certain level.

In this case, the medium supply part (140,240) can be disposed inside the carbon dioxide supply chamber (112), and as an example of the rest of the configuration except for the medium supply part (140,240), the cell culture part (120,220,320,420) and the pump (150) are disposed in the inner space (S) of the constant temperature chamber (111), but they may be arranged to be located on the outside the carbon dioxide supply chamber (112).

Here, the constant temperature chamber (111) may be provided with an air conditioning system that maintains an internal temperature at a constant temperature, and the carbon dioxide supply chamber (112) may include a carbon dioxide supply means that maintains a constant carbon dioxide concentration therein.

Accordingly, the incubator (210) of FIG. 2 can stably supply the carbon dioxide required for cell culture compared to the incubator (110) of FIG. 1 even if the concentration of carbon dioxide is uniformly maintained only in a relatively narrow space limited to the inside of the carbon dioxide supply chamber (112).

Through this, the large-scale cell culture system (200) including the incubator (210) of FIG. 2 can maintain the concentration of the carbon dioxide to be supplied to the medium supply part (140,240) more uniformly compared to the large-scale cell culture system (100) including the incubator (110) of FIG. 1. For this reason, the medium supplied from the medium supply part (140,240) to the cell culture part (120,220,320,420) may have a more uniform carbon dioxide concentration.

Multiple supporters (130, 230) for cell cultivation are disposed inside the cell culture part (120,220,320,420), and thus, the cell culture part (120,220,320,420) may provide a space in which the cells attached to the multiple supporters (130, 230) are cultured, and be connected to the medium supply part (140,240) through connection pipes (161,162,163).

Here, the cells to be cultured may be attached to the multiple supporters (130,230), and the cells attached to the multiple supporters (130,230) may receive nutrients from the medium filled in the accommodation space (S1) to be described later.

Through this, the cells attached to the multiple supporters (130,230) can be smoothly cultured through the nutrients supplied from the medium.

At this time, the multiple supporters (130,230) may be provided in a plate shape having a predetermined area, and at least some of the multiple supporters (130,230) formed in a plate shape may be arranged in multiple stages in the accommodation space (S1) while being spaced apart from the other supporters (130,230).

Accordingly, the cell culture part (120,220,320,420) according to an embodiment of the present invention may culture a large amount of cells through a single culture process by increasing the degree of integration of the supporters (130,230) disposed in the accommodation space (S1).

In addition, the cell culture part (120,220,320,420) according to an embodiment of the present invention can cultivate a large number of cells while reducing the size of the entire facility by arranging the multiple supporters (130,230) in a single device with multi-stage arrangements.

To this end, the multiple supporters (130,230) can be formed with various materials used in publicly known cell culture may be used without limitation, as long as they are implemented in the form of a plate and are easily attached to cells.

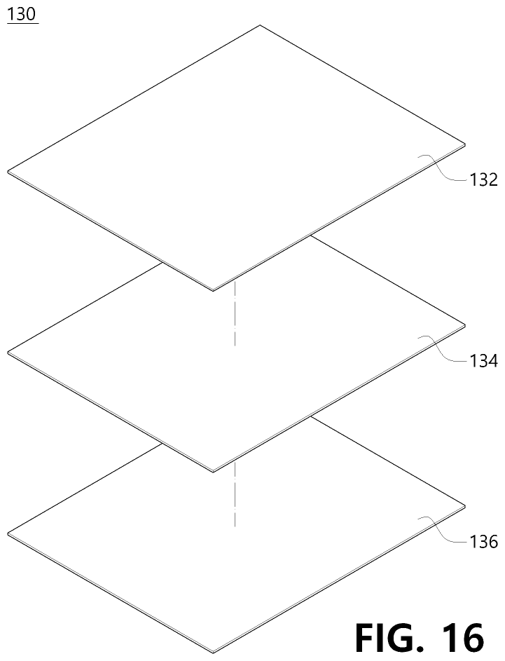

As a non-limiting example, the multiple supporters (130,230) may include a nanofiber membrane (132) in which nanofibers are formed in a three-dimensional network structure through electrospinning. In this case, the supporter (130) may have the three-layer structure which further includes a supporter member (136) attached to one surface of the nanofiber membrane (132) via an adhesive layer (134), in addition to the nanofiber membrane (132) as shown in FIG. 16.

Here, the supporter member (136) may be a plate-shaped film member, and may support one surface of the nanofiber membrane (132). Through this, even if the nanofiber membrane (132) is formed in a flexible plate shape, it can be supported through the support member (136), thereby bending or sagging can be prevented. Accordingly, the supporters (130,230) disposed in the accommodation space (S1) of the cell culture part (120,220,320,420) can maintain the unfolded state, so that cells can be cultured smoothly.

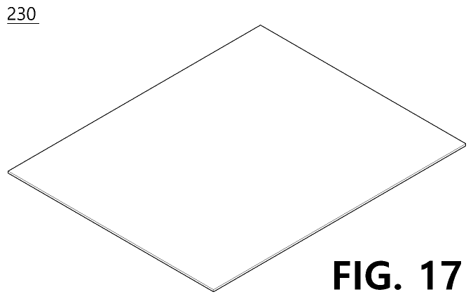

As another example, as shown in FIG. 17, the supporter (230) may be formed of a plate-shaped film member having a predetermined area.

At this time, the supporters (130,230) may have a surface modified so that the cells to be cultured are smoothly attached. As an example, if the supporter (130) includes a nanofiber membrane (132), the nanofiber membrane (132) may be a membrane on which the surface of the nanofiber is motif coated. In addition, when the supporter (230) is provided as a plate-shaped film member, the film member may be a plasma-treated film member.

Accordingly, the cells to be cultured can be smoothly attached to the surface of the supporters (130,230), and the cells to be cultured may be cultured through the nutrients supplied from a medium while attached to the surface of the supporters (130,230).

However, the types of the supporters (130,230) are not limited thereto, and various materials used in publicly known cell culture may be used as long as they can be implemented in the form of plate-shapes and are easily attached to cells.

Such a cell culture part (120,220,320,420) may be disposed in the inner space (S) of the incubator (110,210), the cell culture part (120,220,320,420) may be composed of multiple parts, and the multiple cell culture parts (120,220,320,420) may be connected to the medium supply part (140,240) respectively, in series or in parallel or in a form in which series and parallel are mixed.

Here, the cell culture part (120,220,320,420) may be disposed in the incubator (110,210) so that one surface of the supporters (130,230) disposed therein is parallel to each other with the bottom surface of the incubator (110,210), and may be disposed in the incubator (110,210) so that one surface of the supporters (130,230) disposed therein is perpendicular to each other with the bottom surface of the incubator (110,210).

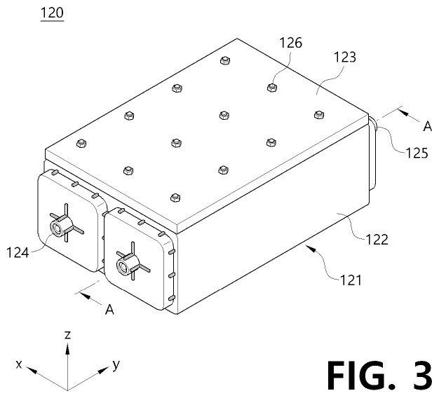

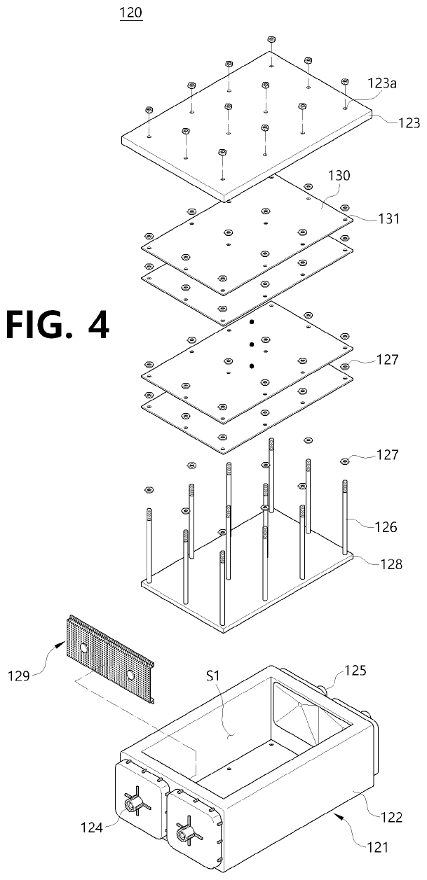

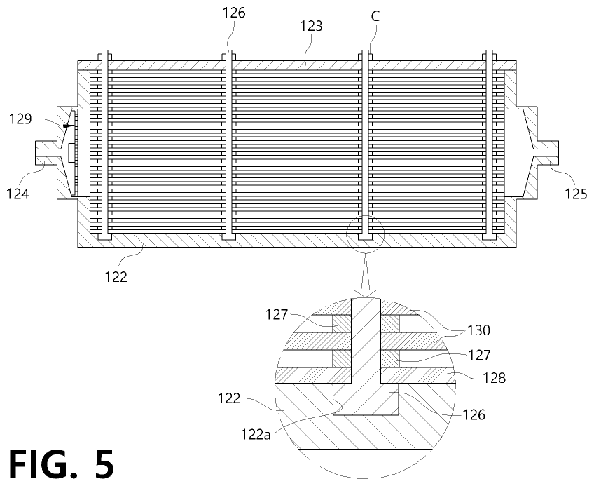

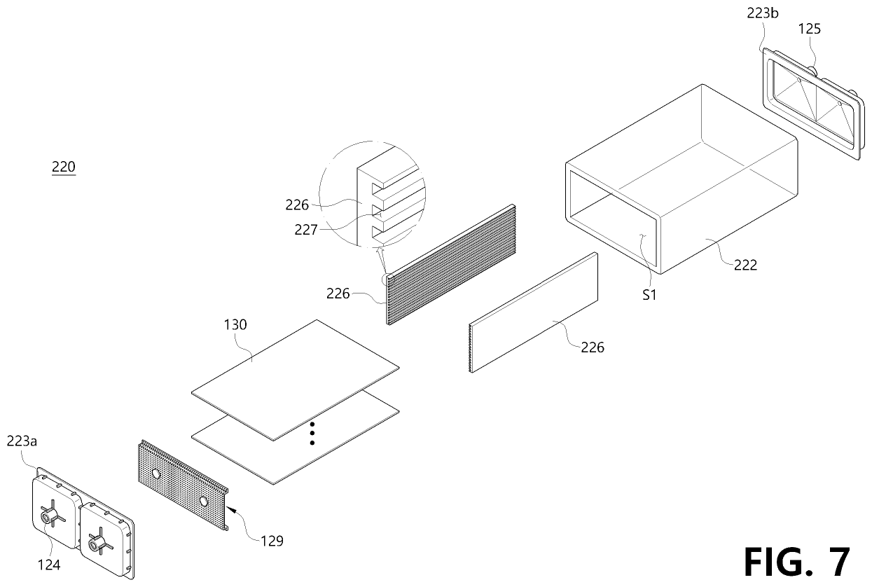

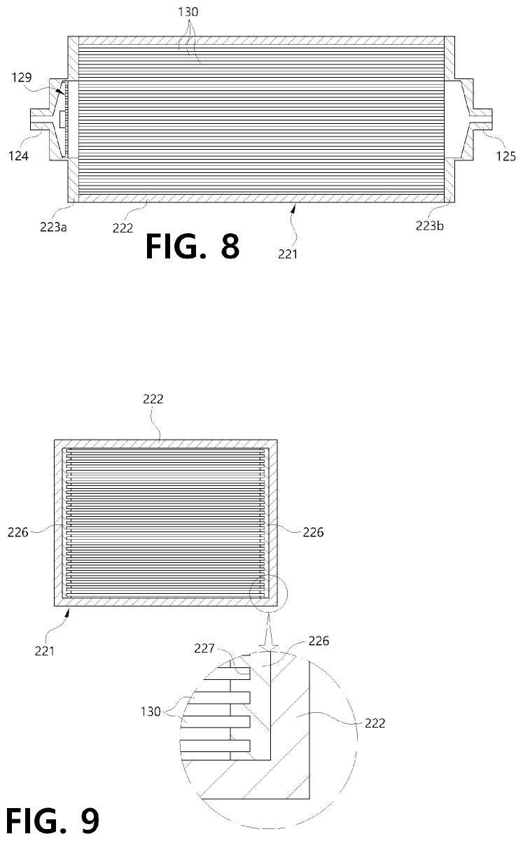

As shown in FIGS. 3 to 15, such cell culture part (120,220,320,420), may include a culture housing (121,221,321,421), the above-mentioned supporters (130,230) and a separated member.

The culture housing (121,221,321,421) may provide a space in which the cells attached to the multiple supporters (130,230) are cultured by placing multiple supporters (130, 230) for cell culture inside.

That is, at least some of the multiple supporters (130,230) may be disposed in the accommodation space (S1) of the culture housing (121,221,321,421) while being spaced apart from each other, and the medium supplied from the outside may be filled in the accommodation space (S1).

To this end, the culture housing (121,221,321,421) may be formed in a hull shape having an accommodation space (S1).

As an example, the culture housing (121) as shown in FIG. 3 to FIG. 5, may include a hull-shaped body (122) having the accommodation space (S1) with an open upper.

In this case, at least a medium inlet (124) and a medium outlet (125) may be formed on the front and rear surfaces of the body (122), respectively, and the accommodation space (S1) with the open upper may be sealed through a cover (123) coupled to the culture housing (121).

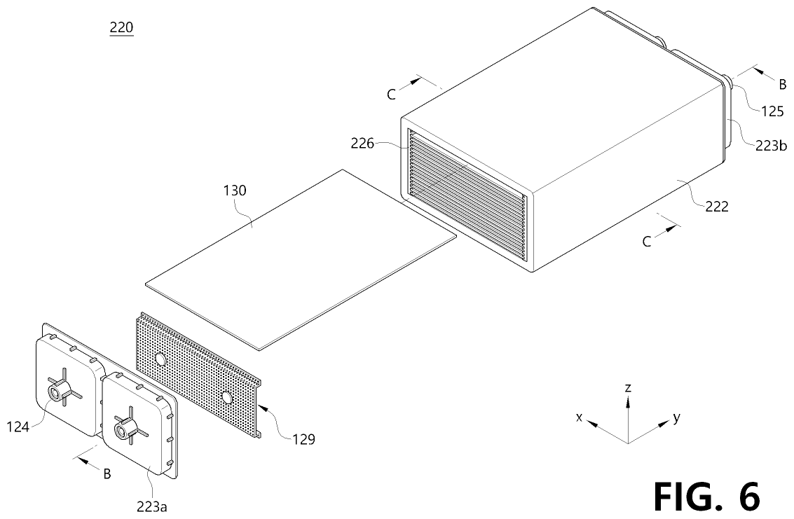

As another example, the culture housing (221,321,421) may include a hull-shaped body (222,322,422) having the accommodation space (S1) with open front and rear surfaces as shown in FIG. 6 to FIG. 15.

In this case, a first cap portion (223a) in which at least one medium inlet (124) is formed and a second cap portion (223b) in which at least one medium outlet (125) is formed may be coupled to the open front and rear surfaces of the bodies (222,322,422), respectively.

Through this, the medium supplied from the outside to the culture housing (121,221,321,421) can fill the accommodation space (S1) through the medium inlet (124), and after the cell culture is completed, the medium filled in the accommodation space (S1) may be discharged to the outside through the medium outlet (125).

Accordingly, the multiple supporters (130,230) disposed in the accommodation space (S1) can be immersed in the medium filling the accommodation space (S1), and the cells attached to each of the supporters (130,230) may receive nutrients necessary for cell culture from the medium.

In the present specification, the width direction, left/right directions and side surfaces of the culture housing (121,221,321,421) may be defined in a direction parallel to the X-axis in FIGS. 3, 6, 10 and 13, and the longitudinal direction, front/rear directions, front/rear surfaces of the culture housing (121,221,321,421) may be defined in a direction parallel to the Y-axis in FIGS. 3, 6, 10 and 13, and the height direction, top/bottom directions, and top/bottom surfaces of the culture housing (121,221,321,421) may be defined in a direction parallel to the Z-axis in FIGS. 3, 6, 10, and 13.

The separated member may be formed in a plate shape and may be disposed in the accommodation space (S1) so as to separate the multiple supporters (130,230) arranged in multiple stages in the accommodation space (S1) from each other.

Through this, the cell culture part (120,220,320,420) according to an embodiment of the present invention may smoothly culture a large amount of cells through a single culture process while increasing the degree of integration of the supporters (130,230) disposed in the accommodation space (S1).

At this time, the separated member may be configured in various forms so that at least some of the supporters (130,230) can be maintained in the state separated with each other by a certain distance while increasing the degree of integration of the multiple supporters (130,230) arranged in multiple stages in the accommodation space (S1).

As an example, as shown in FIGS. 3 to 5, the separated member may be configured in a stacked type so that multiple supporters (130,230) may be arranged in multiple stages along the height direction of the culture housing (121).

That is, the separated member may include multiple supporting bars (126) having a predetermined length and multiple spacers (127) formed in a ring shape, and the multiple supporters (130,230) may be inserted into each of the supporting bars (126).

Specifically, the multiple supporting bars (126) may be spaced apart from each other at a predetermined distance in the accommodation space (S1), and the lower ends of the multiple supporting bars (126) may be fixed respectively to the plate-shaped supporting plate (128) having a predetermined area.

Accordingly, the multiple supporting bars (126) of which lower ends are fixed to the supporting plate (128) may be kept spaced apart from each other.

At this time, the multiple supporting bars (126) may be inserted into the accommodation space (S1) while the lower ends are fixed to the supporting plate (128), respectively. Through this, the multiple supporting bars (126) may be protruded with a predetermined height from the bottom surface on the accommodation space (S1) of the culture housing (121).

In such a state, the multiple supporters (130,230) may be respectively inserted into the supporting bars (126) through multiple passing holes (131) formed through at positions corresponding to the multiple supporting bars (126).

At this time, the multiple spacers (127) having a predetermined height may be inserted into the multiple supporting bars (126), respectively. That is, the multiple spacers (127) and multiple supporters (130,230) may be alternately fastened to each supporting bar (126). Accordingly, the spacer (127) may be disposed between the two supporters (130,230) arranged along the height direction of the culture housing (121), respectively.

Through this, the multiple supporters (130,230) may maintain a plate shape through the multiple supporting bars (126), and the two supporters (130,230) arranged in the vertical direction may maintain a separated distance from each other through the spacer (127). Accordingly, both sides of the supporters (130,230) arranged in the upper and lower directions may be smoothly contacted with the medium filled in the accommodation space (S1).

Here, a setting groove (122a) that is inserted in a predetermined depth into a position corresponding to the multiple supporting bars (126) may be formed on the bottom surface of the culture housing (121), and the cover (123) may have a passing hole (123a) formed through at a position corresponding to the multiple supporting bars (126).

Accordingly, if the multiple supporting bars (126) are inserted into the accommodation space (S1), the lower end of the supporting bar (126) may be inserted into the setting groove (122a), and the upper end of the supporting bar (126) may be exposed to the outside through the passing hole (123a) in the state in which the cover (123) covers the upper portion of the accommodation space (S1), and the upper end of the supporting bar (126) exposed to the outside through the passing hole (123a) may be fastened with a fastening member (C) such as a nut.

Through this, in the cell culture part (120), the opened upper portion of the accommodation space (S1) may be sealed through the cover (123) in the state in which the multiple supporters (130,230) and the spacer (127) are alternately fastened to the multiple supporting bars (126), and because the upper end of the supporting bar (126) protrudes to the outside of the cover (123), and then, it is fastened with the fastening member (C), the cell culture part can maintain the sealed state.

In this embodiment, the plate-shaped supporting plate (128) fixing the lower end of the multiple supporting bars (126) may be omitted. In addition, it is shown that the culture housing (121) has an upper portion opened and the opened upper portion is sealed through a separate cover (123), but is not limited thereto, and the culture housing (121) and the cover (123) may be integrally formed.

As another example, the separated member may be configured in a slot type, as shown in FIGS. 6 to 9.

That is, the separated member may have a predetermined length and include two guide members (226) inserted into the accommodation space (S1) along the longitudinal direction of the culture housing (221), and both ends of the multiple supporters (130,230) may be supported by slidingly inserted into the two guide members (226), respectively.

Specifically, the two guide members (226) may be inserted into the accommodation space (S1) so that one surface faces each other, and the two guide members (226) may include multiple slot grooves (227) that are recessed on opposite surfaces facing each other in the longitudinal direction. Here, the multiple slot grooves (227) may be formed to be spaced apart at predetermined distances along the height direction of the guide member (226).

At this time, the two guide members (226) may be disposed so that two inner surfaces of the culture housing (221) that face each other and one surface of the culture housing (221) are in contact with each other. Through this, the two guide members (226) may be disposed in the accommodation space (S1) so that the surfaces on which the slot grooves (227) are formed face each other, and the opposite surface on which the slot groove (227) is not formed may be disposed to contact two inner surfaces facing each other among the inner surfaces of the culture housing (221), respectively.

Accordingly, in the state in which the two guide members (226) are inserted into the accommodation space (S1), when both ends of each of the supporters (130,230) are inserted into the slot grooves (227) formed in the two guide members (226), respectively, both ends of the supporters (130,230) may be supported by the two guide members (226).

Accordingly, each of the supporters (130,230) can be disposed in the accommodation space (S1) in a horizontal state by constraining both ends through the slot groove (227), thereby maintaining a plate shape, each of the supporters (130,230) disposed along the height direction of the culture housing (221) may maintain the state separated apart by the distance between the two slot grooves (227) formed along the height direction of the guide member (226).

Through this, both sides of the multiple supporters (130,230) arranged in multiple stages in the accommodation space (S1) may smoothly contact the medium filled in the accommodation space (S1), and the cells attached to the supporters (130,230) may be smoothly cultured through the nutrients supplied from the medium.

Thus, since the multiple supporters (130,230) can be coupled to the guide member (226) through a sliding method, the cell culture part (220) according to the present embodiment may increase assembly convenience. In this case, the multiple supporters (130,230) may be inserted into the accommodation space (S1) at once using a separate jig or temporary fixture while being fixed to the jig or temporary fixture.

On the other hand, when the separated member is composed of a guide member (226) including a slot groove (227), at least above-described three guide members may be used to further increase the degree of integration.

That is, as shown in FIGS. 10 to 12, the cell culture part (320) according to an embodiment of the present invention may include four guide members (226) having multiple slot grooves (227) formed along the length direction on one surface, and the four guide members (226) may be disposed in the accommodation space (S1) to form a pair with each other.

Specifically, the four guide members (226) may include two of the first guide members (226a) disposed so that one surface and the inner surface of the culture housing (321) are in contact with each other, and two of the second guide members (226b) disposed between above-described two of the first guide members (226a), and the first guide member (226a) and the second guide member (226b) facing each other may form a pair with each other.

Through this, the multiple supporters (130,230) may be disposed in a horizontal direction by two along the width direction of the culture housing (321) through four guide members (226) configured to form a pair with each other, and the multiple supporters (130,230) as in the above-described embodiment, may be arranged in multiple stages along the up and down directions through the slot groove (227) formed in the guide member (226).

Accordingly, the cell culture part (320) according to the present embodiment can further increase the degree of integration of the supporters (130,230), and can cultivate a larger number of cells in large quantities. In this case, two of the second guide members (226b) may be provided as one member having slot grooves (227) each formed on both surfaces thereof.

However, the total number of the guide member (226) is not limited thereto, and four or more guide members (226) may be used depending on the total number of the supporters (130,230), and any way in which they are paired can be used without limitation. In addition, the accommodation space (S1) may also be divided into multiple spaces so that the two guide members (226) forming a pair can be individually inserted in pairs.

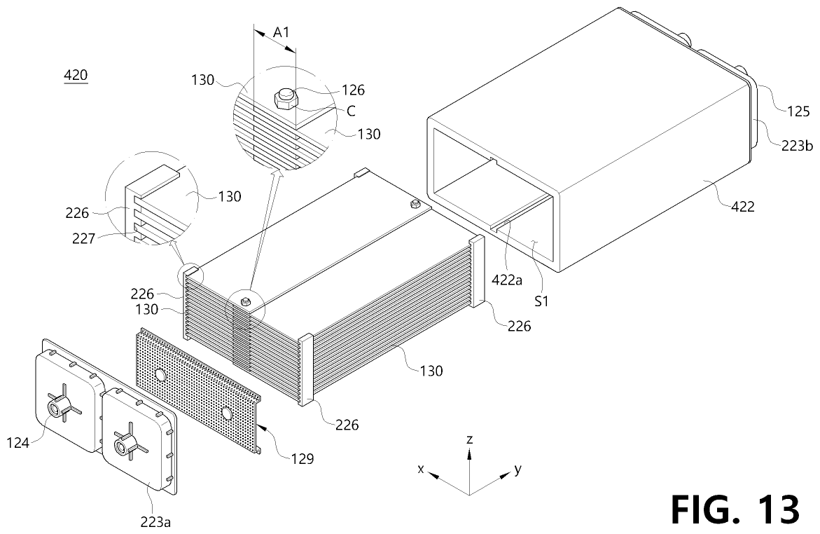

As another example, the separated member may be configured in a combination of a stacking method and a slot method, as shown in FIGS. 13 to 15.

That is, the separated member may be in a form in which the supporting bar (126) shown in FIGS. 3 to 5 and the guide member (226) shown in FIGS. 6 to 9 are combined with each other.

Specifically, the separated member may include four guide members (226) having multiple slot grooves (227) formed on one surface thereof and two supporting bars (126).

In this case, the four guide members (226) may be inserted into the accommodation space (S1) so that the surfaces on which the multiple slot grooves (227) are formed face each other, and the four guide members (226) may be disposed so that one surface thereof and two inner surfaces of the culture housing (421) facing each other contact each other.

In addition, the two supporting bars (126) may be disposed in the accommodation space (S1) so that one surface thereof is positioned between the two guide members (226) facing each other.

Through this, the multiple supporters (130,230) may be inserted into the slot groove (227) formed in the guide member (226) at two corners of the four corners, the remaining two corners may be fitted into the supporting bar (126), respectively.

Accordingly, the multiple supporters (130,230) may be supported through the slot groove (227) formed in the guide member (226) and the supporting bar (126).

Here, the culture housing (421) may be formed with a mounting groove (422a) that is recessed into a predetermined depth so that the two supporting bars (126) can be slidably inserted in the top and bottom sides facing each other.

At this time, the multiple supporters (130,230) may have two supporters (130,230) disposed along the width direction of the culture housing (421), and the two supporters (130,230) may be arranged along the width direction of the culture housing (421) so as to form an overlapping portion (A1) in which a certain area including an end overlaps each other.

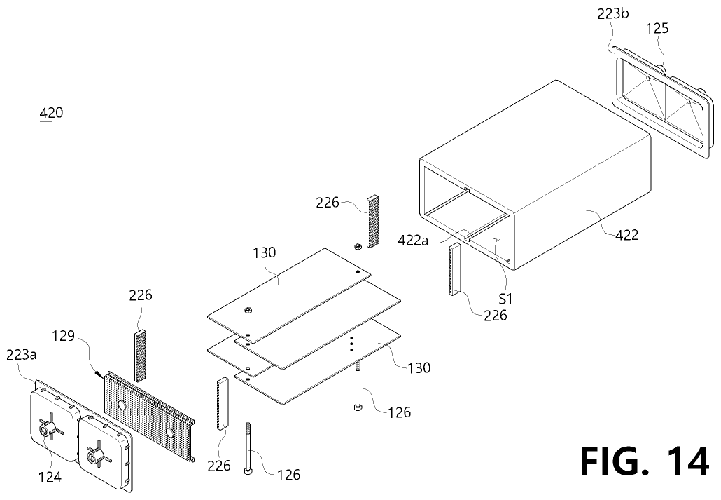

That is, the multiple supporters (130,230) may be stacked in a zigzag manner so that one ends overlap by a predetermined area and directly adhere to each other, as shown in FIGS. 13 and 14, and the other ends that do not overlap each other may be inserted into the slot grooves (227) respectively formed in the four guide members (226).

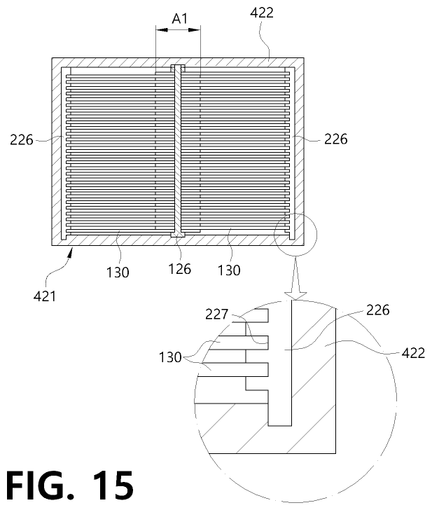

In addition, the two supporting bars (126) may be disposed to pass through the overlapping portion (A1) in which the supporters (130,230) overlap each other by a predetermined area, as shown in FIGS. 13 and 15.

Accordingly, the multiple supporters (130,230) overlap each other and the directly stacked portion may be fastened to the supporting bars (126), and the remaining two corners that do not overlap each other may be supported through the guide member (226).

In this case, the multiple supporters (130,230) may be inserted into the accommodation space (S1) of the culture housing (421) in the state of the assembly in which the four guide members (226) are coupled to the corner through the slot groove (227), and the two supporting bars (126) are coupled to the overlapping portion (A1).

Through this, the multiple supporters (130,230) may maintain a plate shape through the overlapping portion (A1) overlapping each other and the guide member (226), and the two supporters (130,230) disposed along the height direction may maintain a separated distance from each other through the thickness of the other supporters (130,230) disposed to partially overlap between the two supporters (130,230). Accordingly, both sides of the two supporters (130,230) adjacent to each other may smoothly contact the medium filled in the accommodation space (S1).

Through this, the multiple supporters (130,230) can be disposed two at a time in a horizontal direction through the four guide members (226) and the two supporting bars (126), thereby further increasing the degree of integration and cultivating a larger number of cells in large quantities.

In addition, in the case of the present embodiment, even if a separate spacer is not used, the degree of integration can be maximized by maintaining the two supporters (130, 230) separated apart from each other.

In this embodiment, the separated member is illustrated and described as being composed of four guide members (226) and two supporting bars (126), but is not limited thereto, and the numbers of the guide member (226) and the supporting bar (126) may be appropriately changed, and as in the above-described embodiment, the guide members (226) may be provided in two so that one side of the supporters (130,230) may be inserted into the slot groove (227).

Meanwhile, as described above, the culture housings (121,221,321,421) may include at least one medium inlet (124) and one medium outlet (125) that introduce the medium supplied from the medium supply part (140,240) into the inside of the accommodation space (S1) or discharge the medium filled in the accommodation space (S1) to the outside.

As described above, such this medium inlet (124) and outlet (125) may be formed directly on the body (122) of the culture housing (121), or may be formed in separate cap parts (223a,223b) that are coupled to the body (222,322,422) of the culture housing (221,321,421).

That is, as shown in FIG. 3 to FIG. 5, when the body (122) of the culture housing (121) is provided in the shape of an enclosure with front, rear and side surfaces sealed, the medium inlet (124) and the medium outlet (125) may be formed directly on the front and rear surfaces of the body (122).

In addition, as shown in FIG. 6 to FIG. 14, when the body (222, 322, 422) of the culture housing (221, 321, 421) are provided in enclosure shapes with open front and rear sides, and are sealed through separate cap parts (223a,223b) coupled to the front and rear sides of the body (222,322,422), the medium inlet (124) and the medium outlet (125) may be formed in the cap parts (223a,223b), respectively.

Through this, the medium supplied from the medium supply part (140,240) to the cell culture part (120,220,320,420) may be discharged to the outside of the accommodation space (S1) through the medium outlet (125) after introducing into the inside of the accommodation space (S1) through the medium inlet (124).

At this time, the inner surface of the cap part (223a) on which the culture medium inlet (124) is formed or the inner surface of the front surface of the body (122) on which the culture medium inlet (124) is formed may be formed such that is recessed inwardly around the medium inlet (124).

That is, the inner surface of the cap part (223a) on which the medium inlet (124) is formed or the inner surface of the front surface of the body (122) on which the medium inlet (124) is formed may be formed to have a conical or square pyramid shape whose cross-sectional area gradually increases along the direction in which the medium moves from the end of the medium inlet (124), and the end of the medium inlet (124) may form a central portion of the conical or square pyramid shape.

In other words, the inner surface of the cap part (223a) on which the culture medium inlet (124) is formed or the inner surface of the front surface of the body (122) on which the culture medium inlet (124) is formed may be formed to be concave toward the direction opposite to the inflow direction of the culture medium around the culture medium inlet (124).

Through this, the medium flowing from the medium supply part (140,240) through the medium inlet (124) may be smoothly introduced into the accommodation space (S1).

At this time, a distributing plate (129,229) may be disposed between the medium inlet (124) and the supporters (130,230) disposed in the accommodation space (S1) in order to disperse the medium introduced through the medium inlet (124), and the distributing plate (129,229) may be disposed to be separated apart from the ends of the supporters (130,230) disposed in the accommodation space (S1) by a predetermined distance.

Such distributing plate (129,229) may prevent the medium introduced from the outside through the medium inlet (124) from moving immediately the inside of the accommodation space (S1).

That is, the medium flowing from the medium supply part (140,240) through the medium inlet (124) may collide with the distributing plate (129,229) and spread evenly.

Through this, the medium evenly distributed in the process of passing through the distributing plate (129,229) may be moved to the space between each of the supporters (130,230) at the same time regardless of the positions of the multiple supporters (130,230) disposed in the accommodation space (S1), so that the medium may be smoothly supplied to the supporters (130, 230).

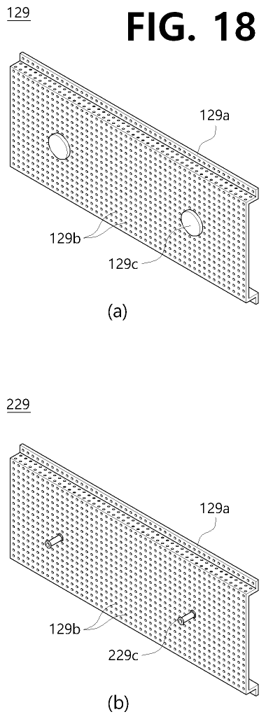

As an example, the distributing plate (129,229) as shown in FIG. 18, may be configured in a form including a plate-shaped body (129a) having a predetermined area and multiple through holes (129b) formed through the body (129a), but is not limited thereto, and may be a plate-shaped mesh net in which multiple through holes are formed.

At this time, the distributing plate (129,229) may include an obstruction means (129c,229c) that blocks the medium introduced from the outside through the medium inlet (124) from moving directly into the inside of the accommodation space (S1).

Such obstruction means (129c,229c) may be formed at a position corresponding to the medium inlet (124).

As an example, the obstruction means (129c) as shown in (a) of FIG. 18, may be a plate-shaped member formed with a predetermined area on one surface of the body (129a) so as to block the medium from passing directly through the body (129a).

Alternatively, the obstruction means (129c) as shown in (b) of FIG. 18, may be a protrusion protruding from the body (129a) toward the medium inlet (124) for a predetermined length. In this case, since one end of the protrusion is located at a close distance to the end of the medium inlet (124), the medium introduced from the medium inlet (124) may hit the end of the protrusion. Through this, the protrusion may more effectively disperse the medium introduced through the medium inlet (124).

The medium supply part (140,240) can store medium containing nutrients necessary for cell culture therein. Such medium supply unit (140, 240) can supply the medium stored therein to the cell culture part (120,220,320,420) by being connected to the cell culture part (120,220,320,420) via the connecting tube (161,162,163).

To this end, as shown in FIGS. 19 to 22, the medium supply part (140,240) may include an enclosure-shaped medium housing (141,241) having a storage space (S2) for storing a certain amount of the medium.

In this case, the medium housing (141,241) may include an inlet (146) and an outlet (145) through which the medium is introduced or discharged in order to supply the medium stored in the storage space (S2) to the cell culture part (120,220,320,420), and then, to recover it.

Here, the inlet (146) may be connected to the medium outlet (125) of the cell culture part (120,220,320,420), and the outlet (145) may be connected to the medium inlet (124) of the cell culture part (120,220,320,420) via a pump (150).

Through this, the medium stored in the storage space (S2) may be recovered toward the medium supply part (140,240) after being supplied to the cell culture part (120,220,320,420) through the operation of the pump (150).

At this time, the medium supply part (140,240) may be disposed in the inside space (S) of the incubator (110), as shown in FIG. 1, or in the carbon dioxide supplying chamber (112) as shown in FIG. 2.

Accordingly, the medium supply part (140,240) may maintain a constant concentration of carbon dioxide dissolved in the medium, and maintain the pH of the medium in a state suitable for cell culture.

Through this, even if the concentration of dissolved carbon dioxide decreases during the process in which the medium moves to the cell culture part (120,220,320,420), and then, it is recovered to the storage space (S2) through the parts inlet (146), the medium may be changed to an appropriate pH required for cell culture through the carbon dioxide introduced from the inner space (S) of the incubator (110) or the carbon dioxide supply chamber (112) after being recovered to the storage space (S2) of the medium supply part (140,240).

Therefore, even if the medium repeatedly circulates the cell culture part (120,220,320,420) and the medium supply part (140,240) through the pump (150), the cells attached to the supporters (130,230) may be cultured smoothly by being able to continuously receive a medium in a state suitable for cultivation.

To this end, the medium housing (141,241) may have the shape of an enclosure with the upper part open so that external carbon dioxide can flow in. At this time, the plate-shaped filter member (143) may cover the open upper portion of the medium housing (141,241), and may be fastened to the medium housing (141,241) through a separate fixing frame (144).

Here, the filter member (143) may be made of a material that allows carbon dioxide to pass while blocking the inflow of foreign substances. Through this, the medium can be prevented from contamination in advance by other foreign substances by blocking the inflow of other foreign substances while receiving carbon dioxide smoothly through the filter member (143).

An appropriate number of the medium supply part (140,240) may be used according to the total number of the cell culture part (120,220,320,420) described above.

At this time, the inlet (146) of the medium supply part (140,240) may be formed in the medium housing (141,241) so as to be located at a relatively higher position than the outlet (145). That is, the outlet (145) may be formed in the medium housing (141,241) to be located at a position relatively closer to the bottom surface of the medium housing (141,241) than the inlet (146), and the inlet (146) may be formed to be located at a position relatively far from the bottom surface of the medium housing (141,241) than the outlet (145).

Through this, the medium flowing from the accommodation space (S1) of the cell culture part (120,220,320,420) to the storage space (S2) through the inlet (146) may move toward the cell culture part (120,220,320,420) through the outlet (145) formed at a relatively low position.

For this reason, even if the medium includes bubbles generated in the process of circulating along the connection pipes (161,162,163) or in the process of being recovered to the storage space (S2) through the inlet (146), the bubbles included in the medium may move upward by buoyancy in the process of moving the medium toward the outlet (145) formed at a position relatively lower than the inlet (146).

Through this, the medium supplied to the cell culture part (120,220,320,420) through the outlet (145) may maintain the state that does not contain air bubbles. Accordingly, the cells attached to the supporters (130,230) may be smoothly supplied with the nutrients from the medium by not being disturbed from the air bubbles.

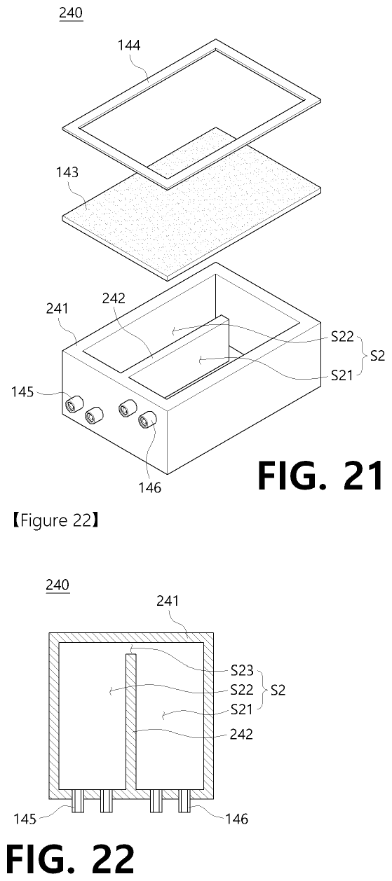

On the other hand, the medium supply part (240) that can be applied to the large-cell culture system (100,200) according to an embodiment of the present invention may divide the storage space (S2) in which the medium is stored into at least two spaces, as shown in FIG. 21 and FIG. 22.

To this end, the medium supply part (240) may include at least one partition wall (242) protruding from the bottom surface of the medium housing (241), the storage space (S2) formed in the medium housing (241) may be divided into a medium recovery space (S21) and a medium supply space (S22) via the partition wall (242).

At this time, the partition wall (242) may protrude from the bottom surface of the medium housing (241), so that its one end is connected to the inner surface of the medium housing (241), and the other end is spaced apart from the other inner surface facing the inner surface of the medium housing (241).

Accordingly, the medium recovery space (S21) and the medium supply space (S22) may communicate with each other through a communication path (S23) formed between the end of the partition wall (242) and the inner surface of the medium housing (241) facing each other.

In this case, the inlet (146) may be formed at a position communicating with the medium recovery space (S21), and the outlet (145) may be formed at a position communicating with the medium supply space (S22). In addition, the outlet (145) may be formed in the medium housing (241) to be located at a position relatively lower than the inlet (146) as described above.

That is, the outlet (145) may be formed in the medium housing (241) to communicate with the medium supply space (S22) while being located at a position relatively closer to the bottom surface of the medium housing (141,241) than the inlet (146), and the inlet (146) may be formed to communicate with the medium recovery space (S21) while being located at a position relatively far from the bottom surface of the medium housing (141,241) than the outlet (145).

Through this, the medium introduced into the medium supply part (240) from the cell culture part (120,220,320,420) may move a relatively longer distance compared to the medium supply part (140) as described above. That is, the medium introduced into the medium supply part (240) may have the increased moving distance until discharged to the outside through the outlet (145) by moving to the medium supply space (S22) through the communication path (S23) after flowing into the medium recovery space (S21).

Accordingly, the medium may have an increased time during which carbon dioxide can be dissolved in the process of moving from the inlet (146) to the outlet (145). In addition, even if the medium contains air bubbles, the air bubbles contained in the medium are completely removed from the medium due to buoyancy in the process of moving from the medium recovery space (S21) to the medium supply space (S22) through the communication path (S23).

Through this, the medium supplied to the cell culture parts (120,220,320,420) through the outlet (145) may maintain the best state that does not contain air bubbles. Accordingly, the cells attached to the supporters (130,230) may be cultured more smoothly.

Thus, the large-cell culture system (100,200) according to an embodiment of the present invention is disposed inside the sealed incubator (110,210) in which the medium supply part (140,240), the pump (150), and the cell culture part (120,220,320,420) are sealed, and configured such that the medium circulates the medium supply part (140, 240) and the cell culture part (120,220,320,420) through the pump (150), thereby being implemented as a closed circulation system.

In addition, the large-cell culture system (100,200) according to an embodiment of the present invention continuously supplies carbon dioxide of a certain concentration through the incubator (110,210), and thus, the medium circulating through the medium circulating the supplying part (140,240), the pump (150), and the cell culture part (120,220,320,420) may be maintained at a constant pH suitable for cell culture.

For this reason, the large-cell culture system (100,200) according to an embodiment of the present invention enables reuse of a medium required for cell culture, so that the production cost can be reduced by minimizing the amount of medium used.

Besides, in the large-scale cell culture system (100, 200) according to one embodiment of the present invention, the cell culture part (120, 220, 320, 420) is configured such that multiple supporters (130, 230) formed in a plate shape having a predetermined area are arranged in multiple stages in the accommodation space (S1). Thus, even if the total size of the cell culture part (120,220,320,420) is reduced, a large number of cells may be attached to the multiple supporters (130,230). Through this, a large number of cells can be stably cultured as well as the size of the entire facility can be implemented in a small size.

Meanwhile, the large-scale cell culture system (100,200) according to an embodiment of the present invention may further include a driving part that rotates the cell culture part (120,220,320,420).

That is, the cell culture part (120,220,320,420) is rotated in the vertical direction through the driving of the driving part, so that one surface of the supporters (130, 230) disposed in the accommodation space (S1) may be disposed in a parallel or vertical state with the bottom surface of the incubator (110,210).

In addition, the cell culture part (120,220,320,420) may allow cells to be evenly attached to the entire area of the supporters (130,230) in the process of attaching the cells contained in the medium to the supporters (130,230) by rotating in the left and right directions through the driving of the driving part.

Such the driving part can control the overall driving through the control part, and the driving part may be configured to enable both the first rotation to rotate the cell culture part (120,220,320,420) around the X-axis and the second rotation around the Z-axis.

Here, the control part can control the overall operation of the entire large-scale cell culture system (100,200) together with the driving part.

To this end, the driving part may include the first motor (181) for the first rotation and the second motor (187) for the second rotation, as shown in FIGS. 23 to 25.

Specifically, the first motor (181) may be installed outside the incubator (110,210), and in the inside of the incubator (110,210), a structure for separating the mounting plate (184) from the bottom surface of the incubator (110,210) may be installed as well as the mounting plate (184) to which the cell culture part (120,220,320,420) is fixed.

That is, at least two supporters (182) having a predetermined height may be installed inside the incubator (110,210), and the rotating table (183) having a predetermined length may be connected to the two supporters (182) so as to be rotatable about the X-axis.

In addition, the mounting plate (184) may be coupled to the rotating table (183) via the coupling part (185), and the rotating table (183) may be connected to the first motor (181) via the pulley (186).

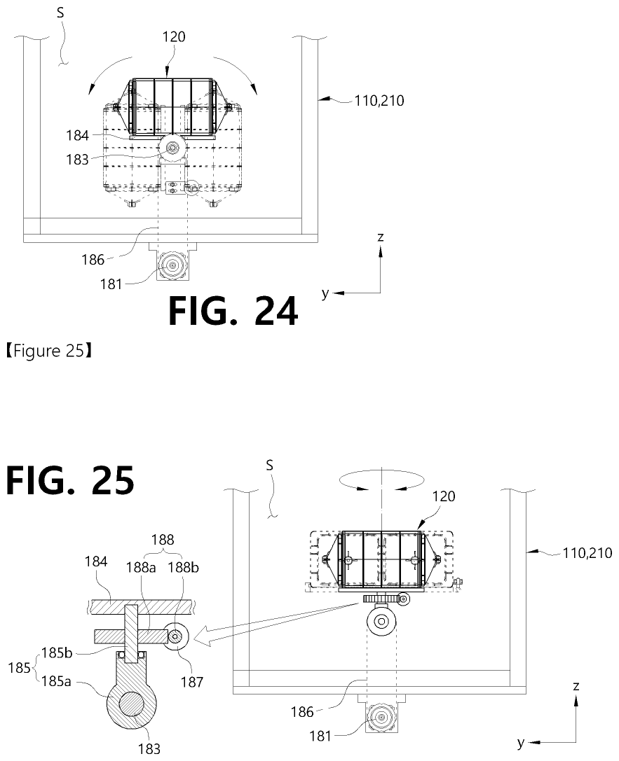

Accordingly, as shown in FIG. 24, when the first motor (181) is driven, the driving force is transmitted through the pulley (186) so that the rotating table (183) can be rotated, and the cell culture part (120,220,320,420) may be rotated around the X-axis through the rotation of the rotary table (183).

At this time, the second motor (187) for rotating the cell culture part (120,220,320,420) around the Z-axis may be installed on the lower side of the mounting plate (184), and the mounting plate (184) may be rotated about the Z-axis through the driving of the second motor (187).

To this end, as shown in FIG. 25, the coupling portion (185) includes the first coupling part (185a) fixedly coupled to the rotating table (183) and the second coupling part (185b) fixedly coupled to the mounting plate (184), and the second coupling part (185b) may be rotatably connected to the first coupling part (185a) about the Z-axis.

Here, the driving force of the second motor (187) may be transmitted to the second coupling part (185b) through the gear part (188) corresponding to each other, and the second coupling part (185b) may be rotated about the Z-axis with respect to the first coupling part (185a) through the driving of the second motor (187). As an example, the gear part (188) may include a worm (188b) axially coupled to the second motor (187) and a worm wheel (188a) fixedly coupled to the second coupling part (185b).

Accordingly, as shown in FIG. 25, when the second motor (187) is driven, the driving force is transmitted through the gear part (188) so that the second coupling part (185b) can be rotated, and the cell culture part (120,220,320,420) can be rotated around the Z-axis through the rotation of the second coupling part (185b).

However, the configuration for rotating the cell culture part (120, 220, 320, 420) is illustrated as above, but is not limited thereto. If the mounting plate (184) can be rotated around the X-axis while being able to rotate around the Z-axis, all of a variety of publicly known methods can be applied.

Meanwhile, the large-scale cell culture system (100,200) according to an embodiment of the present invention can automatically perform the cell culture process including cell attachment, cell cultivation, and cell recovery.

To this end, in the large-scale cell culture system (100,200) according to an embodiment of the present invention as shown in FIGS. 1 and 2, the cell culture part (120,220,320,420), the pump (150) and the medium supply part (140,240) may be connected via multiple connection pipes (161, 162, 163), and the medium may repeatedly circulate through the cell culture part (120,220,320,420) and the medium supply part (140,240) through the driving of the pump (150).

In addition, the multiple opening and closing valves (171,172,173) may be provided at the multiple connection pipes (161, 162, 163), and the first supply line (164), the second supply line (165), and the emission line (166) may be connected to the multiple connection pipes (161,162,163).

Specifically, the outlet (145) may be connected to the pump (150) through the first connection pipe (161) in the medium supply part (140,240), and the pump 150 may be connected to the medium inlet (124) of the cell culture part (120,220,320,420) via the second connection pipe (162) In addition, the medium outlet (125) of the cell culture part (120,220,320,420) may be connected to the inlet (146) of the medium supply part (140,240) via the third connection pipe (163).

At this time, the first opening/closing valve (171) may be provided on the first connection pipe (161), the second opening/closing valve (172) may be provided on the second connection pipe (162), and the third opening/closing valve (173) may be provided on the third connection pipe (163).

In this state, the second connection pipe (162) may be connected to the first supply line (164) between the second opening and closing valve (172) and the cell culture part (120,220,320,420) via the fourth opening and closing valves (174). In addition, the second connection pipe (162) may be connected to the second supply line (165) between the second opening and closing valve (172) and the cell culture part (120,220,320,420) via the fifth opening and closing valve (175). Besides, the third connection pipe (163) may be connected to the emission line (166) between the third opening and closing valve (173) and the medium supply part (140,240) via the sixth opening and closing valve (176), the first supply line (164) or the second supply line (165) may be connected to the gas supply line (167) via the seventh opening/closing valve (177).

Here, the first supply line (164) supplies to the cell culture part (120,220,320,420) the washing liquid or a medium containing the cells to be cultured, and the second supply line (165) may supply trypsin for chemically separating the cells attached to the supporters (130,230) from the supporters (130,230), and the gas supply line (167) may supply high-pressure gas to the cell culture part (120,220,320,420).

In addition, the sensor (178) may be disposed on the third connection pipe (163) between the third opening/closing valve (173) and the medium outlet (125) of the cell culture part (120,220,320,420), and the multiple opening/closing valves and the sensor described above may be electrically connected to the control part.

At this time, the first opening/closing valve (171), the second opening/closing valve (172) and the third opening/closing valve (173) may be NC valves, and the fourth opening/closing valve (174), the fifth opening/closing valve (175) and the sixth opening/closing valve (176) may be NO valves. Through this, it is possible to minimize the opening and closing operations of the opening and closing valves.

Here, the cell culture part (120,220,320,420) may be disposed so that one surface of the supporters (130, 230) is horizontal with respect to the bottom surface of the incubator (110,210) during cell culture, but one surface of the supporters (130,230) may be disposed to be vertical with respect to the bottom surface of the incubator (110,210).

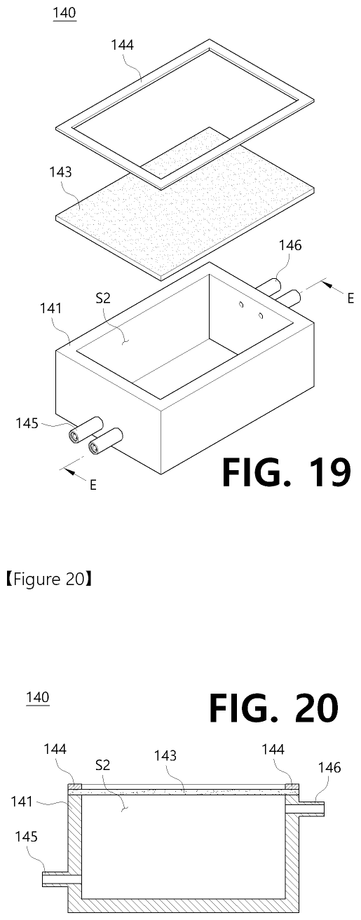

Hereinafter, for convenience of description, it will be described that in the cell culture part (120,220,320,420), the medium inlet (124) and the medium outlet (125) are provided to locate on opposite sides of the culture housing (121,221,321,221), and the medium inlet (124) faces downward in the process of culturing the cells, and the supporters (130,230) accommodated in the accommodation space (S1) are disposed so that one surface thereof is perpendicular to the bottom surface of the incubator (110,210). In addition, it will be described that the medium supply part (140) shown in FIG. 19 and FIG. 20 is used.

First, a certain amount of medium is stored in the storage space (S2) of the medium supply part (140), and the cell culture part (120,220,320,420) is rotated about the X-axis through the driving of the first motor (181), so that one surface of the supporters (130,230) is disposed to be in a vertical state with respect to the bottom surface of the incubator (110,210) while the medium inlet (124) faces downward (refer to the hidden line in FIG. 24).

In this state, the second opening/closing valve (172) maintains a closed state and the fourth opening/closing valve (174) maintains an open state. Then, the medium containing the cells to be cultured is supplied to the cell culture part (120,220,320,420) through the first supply line (164).

Accordingly, the cells contained in the medium move to the accommodation space (S1) of the cell culture part (120,220,320,420) together with the medium, and then are attached to the respective supporters (130,230).

At this time, if the medium supplied through the first supply line (164) completely fills the accommodation space (S1) of the cell culture part (120,220,320,420), and then flows into the third connection pipe (163), the control part detects that the medium flows into the third connection pipe (163) through the sensor (178).

In this case, the control part stops the supply of the medium supplied from the first supply line (164) by changing the third opening and closing valve (173) and the fourth opening and closing valve (174) to a closed state.

Here, when the supply of the medium from the first supply line (164) is stopped, the cell culture part (120,220,320,420) may be rotated around the X-axis by driving the first motor (181) so that one surface of the supporters (130,230) is in a state parallel to the bottom surface of the incubator (110,210) in order for stably attaching the cells to each of the supporters (130, 230) (Refer to the solid lines in FIGS. 23 and 24).

At this time, the cell culture part (120,220,320,420) may be rotated in a forward or reverse direction around the Z-axis through the driving of the second motor (187) so that the medium filled in the accommodation space (S1) can be spread evenly over the entire area of the supporters (130,230).

Accordingly, the cells included in the medium may be evenly adhered over the entire area of the supporters (130, 230) instead of being concentrated on the partial area of the entire area.

Then, when the cells are stably attached to the supporters (130,230), the cell culture part (120,220,320,420) are rotated about the X-axis through the driving of the first motor (181) so that the medium inlet (124) is changed to its original state facing downward (Refer to the hidden line in FIG. 24).

Here, the fourth opening/closing valve (174) maintains a closed state, and the first opening/closing valve (171) and the second opening/closing valve (172) are changed to open states.

Accordingly, the medium stored in the medium supply part (140,240) may be circulated between the medium supply part (140,240) and the cell culture part (120,220,320,420) through the driving of the pump (150).

At this time, the medium supplied to the cell culture part (120,220,320,420) from the medium supply part (140,240) and then recovered to the medium supply part (140,240) may be resupplied to the cell culture part (120,220,320,420) after being changed to a pH suitable for cell culture through the introduction of carbon dioxide. Through this, the cells attached to the supporters (130,230) can be cultured smoothly by being continuously supplied with a medium having a pH suitable for culture.

Then, when the cultivation of the cells attached to the supporters (130,230) is completed, the second opening/closing valve (172) is changed to a closed state, and the sixth opening/closing valve (176) is changed to an open state. In this state, high-pressure gas may be supplied to the cell culture part (120,220,320,420) through the gas supply line (167). Through this, the medium filled in the cell culture part (120,220,320,420) is discharged to the outside through the emission line (166) after moving along the third connection pipe (163) through the high-pressure gas.

At this time, the medium outlet (125) may be disposed to face downward by rotating the cell culture part (120,220,320,420) around the X-axis through the driving of the first motor (181). Through this, the medium filled in the cell culture part (120,220,320,420) may be smoothly discharged to the outside through the emission line (166) after moving along the third connection pipe (163) through the high-pressure gas.

Thereafter, when the medium filled in the accommodation space (S1) of the cell culture parts (120,220,320,420) is completely removed, the first opening/closing valve (171) is changed to an open state, and the washing liquid is supplied to the cell culture part (120,220,320,420) through the first supply line (164). Accordingly, the washing liquid is discharged to the outside through the emission line (166) through the third connection pipe (163) after washing the multiple supporters (130,230) mounted on the cell culture part (120,220,320,420).

In this case, the culture medium inlet (124) may be disposed downward, and the medium outlet (125) may be disposed downward in the cell culture part (120,220,320,420).

Then, in the state in which the cell culture part (120,220,320,420) is changed so that the medium inlet (124) faces downward, the fourth opening/closing valve (174) is changed to a closed state, and the fifth opening/closing valve (175) is opened.

Accordingly, trypsin may be supplied to the cell culture part (120,220,320,420) through the second supply line (165), and the trypsin supplied to the cell culture part (120,220,320,420) through the second supply line (165) may chemically separate the cells attached to the supporters (130,230).

Then, when the cell culture part (120,220,320,420) is separated and recovered from the large-scale cell culture system (100,200), the cells separated from the supporters (130,230) can be recovered in large quantities.

As mentioned above, embodiments of the present invention have been described above, but the ideas of the present invention are not limited to the embodiments presented in this specification, and those skilled in the art who understand the ideas of the present invention will be able to easily propose other embodiments by adding, changing, deleting, or supplementing components within the scope of the same ideas, but these will also be said to fall within the scope of the present invention.

1. A large-scale cell culture system comprising:
an incubator that includes an inner space to provides a culture environment in which a cell is stably cultured;
a cell culture part that is disposed in the inner space and includes multiple supporters for cell culture disposed therein;
a medium supply part that is disposed in the inner space and stores a predetermined amount of medium supplied to the cell culture part; and
a pump that is disposed in the inner space, and is connected to the cell culture part and the medium supply part through a connection pipe, respectively, and circulates the medium so that the medium stored in the medium supply part is recovered to the medium supply part after being supplied to the cell culture part,
wherein the multiple supporters are provided in a plate shape having a predetermined area, and are arranged in a state separated apart from each other at a predetermined distance along a height direction inside the cell culture part.

2. The large-scale cell culture system of claim 1, wherein the supporter includes:
a plate-shaped nanofiber membrane that is coated with a protein motif; and
a support member that is attached to one surface of the nanofiber membrane through an adhesive layer to support the nanofiber membrane.

3. The large-scale cell culture system of claim 1, wherein the supporter is a plasma-treated plate-shaped film member.

4. The large-scale cell culture system of claim 1, wherein the inner space is formed as one inner space in which a temperature is maintained at a constant temperature while a carbon dioxide concentration is maintained at a certain level, and
the cell culture part and the medium supply part are disposed together in the one inner space.

5. The large-scale cell culture system of claim 1, wherein the incubator includes:
a constant temperature chamber that maintains a temperature of the inner space at a constant temperature, and in which the cell culture part and the pump are disposed; and
a carbon dioxide supply chamber that is disposed inside the constant temperature chamber and maintains the internal carbon dioxide concentration at a certain level, and
the medium supply part is disposed inside the carbon dioxide supply chamber.

6. The large-scale cell culture system of claim 1, wherein the cell culture part includes:
an enclosure-shaped culture housing having an accommodation space filled with the medium;
the multiple supporters that are arranged in multiple stages in the accommodation space so that a cell is cultured; and
a separated member that separates between two supporters facing each other so that the multiple supporters are separated apart from each other along a height direction of the culture housing.

7. The large-scale cell culture system of claim 6, wherein the separated member includes:
multiple supporting bars that have a predetermined length and are separated apart from each other in the accommodation space; and
a spacer that is respectively fastened to the multiple supporting bars so that two adjacent supporters are separated apart from each other.

8. The large-scale cell culture system of claim 6, wherein the separated member includes at least two guide members that are inserted in the accommodation space so that one surface faces each other, and
the two guide members include multiple slot grooves that are recessed along a length direction so that each end of the supporter is slidably inserted on one surface facing each other.

9. The large-scale cell culture system of claim 6, wherein the separated member includes:
at least two guide members that are inserted in the accommodation space so that one surface faces each other, and includes multiple slot grooves that are recessed along a length direction so that each end of the supporter is slidably inserted on one surface facing each other; and
at least one supporting bar that is provided to have a predetermined length and disposed in the accommodation space so as to be positioned between the two guide members.

10. The large-scale cell culture system of claim 9, wherein the multiple supporters include a first supporter and a second support that are arranged to overlap each other with a partial area having an end portion along a width direction of the culture housing,
the first supporter is disposed such that one end is inserted into the slot groove of a first guide member among the two guide members, and the other end overlaps with the second supporter,
the second supporter is disposed such that one end is inserted into the slot groove of a second guide member among the two guide members, and the other end overlaps with the first supporter, and
the supporting bar is disposed to pass through an overlapping portion where the first supporter and the second supporter overlap each other.

11. The large-scale cell culture system of claim 6, wherein the culture housing includes at least one medium inlet that is provided on one surface of the culture housing to allow the medium supplied from the medium supply part to flow into the accommodation space, and
an inner surface of the culture housing at the portion in which the medium inlet is formed to be concave toward a direction opposite to an inflow direction of the medium around the center of the medium inlet.

12. The large-scale cell culture system of claim 11, wherein a distributing plate is disposed between the medium inlet and the supporter disposed in the accommodation space so that the medium introduced through the medium inlet is dispersed.

13. The large-scale cell culture system of claim 12, wherein the distributing plate includes:
a plate-shaped body with a predetermined area;
multiple through holes that are formed through the body; and
an interrupting means that is formed at a position corresponding to the medium inlet to prevent the flow of the medium introduced from the medium inlet.

14. The large-scale cell culture system of claim 13, wherein the interrupting means is a protrusion that is formed to protrude in a predetermined length from the body toward the medium inlet or a plate-shaped member that is formed in a predetermined area on one surface of the body.

15. The large-scale cell culture system of claim 1, wherein the medium supply part includes:
a medium housing that is in an enclosed shape with an open upper portion, and includes a storage space in which a certain amount of the medium is stored; and
an inlet and an outlet through which the medium flows in or out so that the medium is returned to the storage space or supplied to the cell culture part, and
the inlet is formed in the medium housing so as to be located at a position relatively higher than the outlet.

16. The large-scale cell culture system of claim 15, wherein the medium housing includes at least one partition wall that protrudes from a bottom surface of the medium housing so that one end is connected to an inner surface of the medium housing and the other end is separated apart from the other inner surface facing the inner surface of the medium housing, and
the storage space is divided into a medium recovery space connected to the inlet and a medium supply space connected to the outlet through the partition wall.

17. The large-scale cell culture system of claim 15, wherein the medium supply part includes a filter member that covers the open upper portion of the medium housing, and the carbon dioxide is supplied to the medium stored in the storage space after passing through the filter member.

18. The large-scale cell culture system of claim 1, further comprising at least one driving part that rotates the cell culture part.

19. The large-scale cell culture system of claim 18, wherein the cell culture part is installed in the inner space of the incubator to enable both a first rotation around an X-axis and a second rotation around a Z-axis through driving of the driving part.
